(19) United States
(12) Patent Application Publication
(10) Pub. No.: US 2007/0244560 A1
(43) Pub. Date: Oct. 18, 2007

(54) Intraocular lens with distortion free valve

(76) Inventors: Alexei Ossipov, Laguna Niguel, CA (US); Thomas R. Paul, Westlake Village, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/402,729

(22) Filed: Apr. 12, 2006

(51) Int. Cl. A61F 2/16

(52) U.S. Cl. 623/6.14

(57) ABSTRACT

The invention provides a medical device and method to reduce the occurrence of pseudophakic pupillary block following implantation of an intraocular lens. The device includes an intraocular lens configured with channels traversing through the surfaces of the lens optic. The invention includes special edge angulations and locations in the optic zone that eliminate optical distortion from channels. The channels perform as valves configured to allow flow of a patient's aqueous humor from one surface of the lens through to the other surface of the lens, thereby preventing fluid pressure from building up in a patient's eye.

### FIELD OF THE INVENTION

The present invention relates generally to implantable intraocular lenses. More specifically, the present invention relates to a distortion-free implantable intraocular lens including a valve configured to permit fluid flow through the lens, from the posterior surface of the lens to the anterior surface of the lens, whereby pseudophakic pupillary block may be prevented.

### BACKGROUND OF THE INVENTION

Patients who undergo ocular surgery with an implantation of an intraocular lens (IOL) are at risk of developing pseudophakic pupillary block. Pupillary block refers to a failure of communication of aqueous humor between the anterior and posterior chambers of an eye caused by obstruction of the pupil and surgical openings in the iris. In pseudophakic pupillary block, the implanted intraocular lens is partly or wholly involved in an obstruction of aqueous flow through the pupil. Pseudophakic pupillary block can develop at any time after the lens implant surgery. If the condition is not recognized and treated early, it can lead to iris bombe, iridocorneal adhesion formation (starting at the periphery and extending toward the center), increasing intraocular pressure, progressive damage to the optic nerve head, and loss of vision. Mean intraocular pressure in a normal population is 15 mm Hg with intraocular pressure in the range of 10 to 21 mm Hg falling within two standard deviations of the mean. Intraocular pressures above 21 mm Hg are considered pathologic.

Pseudophakic pupillary block is caused by a mechanical closure of the pupil by the optic of the implanted artificial intraocular lens or by the development of synechiae between the iris and the artificial lens or remaining lens capsule. Pseudophakic pupillary block also can occur in patients with artificial intraocular lenses implanted in the anterior chamber, either by direct blocking of the pupil by the optic or by the development of adhesions between the vitreous and the posterior iris. Closure of an existing peripheral iridectomy or an absence of a peripheral iridectomy may be a precipitating factor.

Extracapsular surgery that precedes the insertion of the implantable artificial intraocular lens may create retention of a large part of the anterior lens capsule, retained lens matter in the formices of the capsular bag, a tear of the posterior capsule, and lens-vitreous mix that are conducive to inflammatory, proliferative, and fibrotic reactions. The inflammatory reactions produce adhesions between the artificial lens and the uveal tissues, particularly the iris.

Sulcus-supported lenses have a tendency to erode the ciliary processes and the ciliary body resulting in a breakdown of the blood-aqueous barrier. The optics of the sulcus-supported lenses have a greater tendency to partial or complete pupillary capture. The fibrous reactions in the capsular bag also can push the optic out of the bag, a process that may lead to pupil capture.

The shallowness of the anterior chamber due to wound leakage, or pooling of aqueous in the vitreous pushing the lens optic forward, can also push the lens optic firmly against the pupil, effectively blocking the forward movement of the aqueous and causing partial or complete pupil capture.

Pediatric patients are particularly vulnerable to pseudophakic pupillary block. Fibrin formation is encountered more often in children. Furthermore, there is a greater tendency for the optic to come out of the small capsular bag and become captured by the pupil. In neonates and young infants, there is a tendency for the iridectomy opening to shrink and ultimately close.

The net result of all these processes is iris bombe, anterior synechiae formation, glaucoma, and an increased resistance to the forward movement of aqueous humor. Pupillary block can occur if a peripheral iridectomy becomes blocked and the pupil closes as a result of the above factors. Pupillary block is particularly prone to occur with use of an artificial intraocular lens implanted in the anterior chamber, an angle-supported lens, or an iris claw (Artisan) lens. In the pupillary area, the initial adhesions are formed between the pupil and the posterior capsule. As iris bombe develops, adhesions form between the anterior surface of the iris and the optic and the haptic of the implanted artificial intraocular lens. The iris bombe may involve the whole iris, but more often it is multioculated.

Pseudophakic pupillary block following implantation of an implantable artificial intraocular lens is not an uncommon condition. The exact incidence is not known, but it occurs more frequently in pediatric patients, especially those who are very young. Failure to relieve the pupillary block can lead to the development of chronic angle closure glaucoma and glaucomatous optic neuropathy with loss of vision.

Surgical procedures are known that may treat pseudophakic pupillary block after it occurs by breaking up the block and reestablishing flow of aqueous humor. Physicians most commonly perform a peripheral iridectomy to open up the obstruction to flow of aqueous humor. These procedures may carry a risk of further complications.

A typical IOL includes an optic, usually having a diameter of about 6 mm, and haptics or fixation members coupled to (or formed with) the optic to fix the optic within the eye in the region of the extracted lens. IOL's are of two basic types, those having a hard or rigid optic formed, for example, of polymethylmethacrylate (PMMA) and those having a deformable optic which is constructed of a deformable material such as silicone, collagen, hydrogel, or an acrylic. Prior to insertion of the IOL the cataract-affected crystalline lens is generally fragmented and removed by phacoemulsification (“phaco”) through a small incision. If a hard IOL is used, the small phaco incision must be enlarged to approximately the diameter of the hard optic, in order to permit the hard optic to be inserted through the incision and much of the advantage of phacoemulsification lens extraction is thereby obviously lost. If a soft IOL is used, it may be injected into position in a patient's eye. One type of IOL can be deformed (e.g., folded or rolled) to pass through a scleral tunnel incision.

Implantable IOLs including valve mechanisms for flow of fluid within the lens have been described. However, known valve mechanisms do not provide flow completely through the lens from the posterior surface of the lens out through the anterior surface of the lens. For example, one known described device is an intraocular lens having a lens portion defining an anterior surface layer and a posterior surface layer where an interior of the lens portion includes an array of deformable cells each defining a volume of a selected fluid therein. Each deformable cell is in substantial engagement with either the anterior or posterior surface layer. Also provided is means for controllably causing fluid flow to alter the volume in at least a portion of the array of deformable cells to thereby controllably deform the anterior or posterior surface layer and alter optical parameters of the lens. Fluid may flow into or out of the deformable cells, but the patient's aqueous humor does not flow through the lens. For this reason, implanting such a lens would not prevent pseudophakic pupillary block.

Another known described device permitting flow within a lens is an IOL that carries an interior phase or layer comprising a pattern of individual transparent adaptive displacement structures. In the exemplary embodiments, the displacement structures are actuated by shape change polymer that adjusts a shape or other parameter in response to applied energy that in turn displaces a fluid media within the lens that actuates a flexible lens surface. Fluid displacement about the interior space of the lens alters the refractive parameters of the structure without a change in net volume. The patient's aqueous humor does not flow through the lens, and therefore this lens would not prevent pseudophakic pupillary block.

Yet another described device providing flow within a lens is an IOL whose focusing performance can be modified after its implantation in the eye without a need for any invasive procedure. The IOL has an optical chamber having at least a flexible region that is deformable under influence of a fluid. The IOL further include a reservoir for storing an optical fluid in fluid communication with the optical chamber, and a valve that regulates the fluid communication between the reservoir and the optical chamber. The lens can also include a pump that is actuated by an external energy source to transfer the optical fluid between the reservoir and the optical chamber to change the amount of fluid in the optical chamber, thereby modifying the focusing performance of the IOL. The patient's aqueous humor does not flow through the lens.

One other known device provides a central channel in the lens optic for flow of air through the lens anterior and posterior surfaces. The channel has a central opening in both the anterior and posterior surface of the lens. The channel is directed at a 90 degree angle to the surface of the lens. The device does not have a valve configured to control the rate of flow through the lens. Furthermore, the central openings in the optic lead to optical distortion in the region where light passes through the central openings.

Furthermore, a plurality of implants to treat glaucoma, for example, shunt devices, are described in the art. The patient's aqueous humor may flow through these glaucoma shunts. However, these are discrete implants that are not a component of known implantable intraocular lens devices. Because known glaucoma shunts are discrete devices that are not part of a lens optic, distortion of vision is not a consideration in the design of those devices.

There is no known implantable intraocular lens device that is configured for the prevention of pseudophakic pupillary block. Hence, those skilled in the art have recognized a need for an IOL configured for the reduced occurrence and/or prevention of pseudophakic pupillary block. Furthermore those skilled in the art have recognized that such an IOL would preferably include a lens that is free from any distortion that may result from fluid channels that may be configured within the lens. Those skilled in the art have also recognized a need for methods of making and implanting such an intraocular lens, the methods providing for the reduced occurrence of the development of pseudophakic pupillary block. The present invention fulfills these needs and others.

### SUMMARY OF THE INVENTION

Briefly and in general terms, the present invention provides a new and improved intraocular lens having a valve that allows for the flow of aqueous humor through an intraocular lens while maintaining distortion-free optics. The invention is configured for fluid flow through the lens, between the posterior and anterior surfaces of the lens, thus assisting to prevent or reduce the occurrence of pseudophakic pupillary block.

The present invention is advantageous because it prevents pseudophakic pupillary block from occurring in the first place, thereby avoiding the need for additional surgeries in a patient who has had implantation of an IOL. Preventing pseudophakic pupillary block prevents both the medical complications and loss of vision that result from a block, as well as those complications that may result from additional surgeries required to open up a pseudophakic pupillary block.

Furthermore, another aspect of the invention is that it provides a distortion-free lens that is configured to permit flow of aqueous humor from the posterior surface of the lens through and out the anterior surface of the lens. In one aspect of the present invention, a soft lens including valves is configured to create pulsatile micro flow of fluid between two wedge-like surfaces during accommodation of an eye. In one embodiment, the valves and openings in the lens are self cleaning and are configured wherein clogging with internal eye debris and scar tissue is minimized.

In accordance with certain aspects of the present invention there is provided an optically clear and distortion free valve for intraocular lenses configured with at least one cut or channel having a specially configured angle that is made completely through an optic zone of the intraocular lens. In one aspect, there is a center symmetry in the positioning and configuration of the channels or cuts. While other types of cuts or vents might permit aqueous humor flow through an intraocular lens, one aspect of the present invention includes special channel angulations and locations in the optic zone that eliminate optical distortion from the cuts. In another aspect, the present invention includes channels in the lens that are arranged symmetrically around a center of the optic portion of the lens. Yet another aspect of the invention is that the cuts or channels are configured at a critical angle that results in light being reflected from the valve, thereby resulting in distortion free optics for the IOL. In at least one other aspect of the present invention, the cuts are made at a critical angle in the range of zero to twenty degrees.

In still another aspect of the present invention, the configuration of the cuts creates an optical trap for light that may be refracted or reflected by the openings in the channels in the lens. The critical angle of the cuts can be calculated based on the anatomy of the human eye and a refractive index of the intraocular lens material. The special angulations of the channels results in light being reflected from the valve, thereby eliminating reflections caused by the cut, thus providing distortion free optics.

In some aspects of the present invention, the channels through the lens are preferably arranged symmetrically around a center of an optic portion of the lens for better optical properties and less distortion. However, in another aspect, the cuts may be asymmetric. In yet another aspect, a slit-like cut may be made mechanically with a sharp knife blade. In still other aspects, the slit-like cuts may be made with a laser. In yet another aspect, the cuts may be made by high pressure water cuts.

In still further aspects of the present invention, the surface of the intraocular lens has surface openings of the channels configured to support a flow of fluid of five to six microliters per minute through the lens. This fluid rate is important in preventing the build up of pathologic intraocular pressure in the eye. In another aspect of the invention, compression of the soft intraocular lens of the invention during accommodation may cause the soft IOL to flex in response to changes of the ciliary muscles that may also promote pulsatile flow through the channel and between the two openings. This is advantageous for self cleaning of the openings.

In yet a further aspect of the present invention, there is provided an intraocular device for implantation in an eye of a patient, including a peripheral zone disposed around an optical zone, the optical zone having an anterior optical zone face and a posterior optical zone face, and the peripheral zone having an anterior peripheral face and a posterior peripheral face. The intraocular device further includes at least one channel disposed in the optical zone, the channel having an anterior opening and a posterior opening. In one aspect of the invention, the channel is disposed at a critical angle that results in light being reflected from the lens. In at least one aspect of the invention, the channel through the optical zone is at an angle in the range of zero degrees to twenty degrees from a line parallel to a horizontal reference line drawn across the circumferential edge of the peripheral zone.

In another aspect, the present invention provides a method of making a distortion-free IOL configured with a valve. The method includes forming a lens from a biocompatible material, the lens including a peripheral zone disposed around an optical zone, the optical zone having an anterior optical zone face and a posterior optical zone face, and the peripheral zone having an anterior peripheral face and a posterior peripheral face and at least one channel formed through the lens to provide for fluid flow through the lens. In one aspect of the invention, the method includes calculating a critical angle for the channel that results in light being reflected from the lens. In another aspect, the method further includes forming at least one channel through the optical zone at an angle in the range of zero degrees to twenty degrees from a line parallel to a horizontal reference line drawn across the circumferential edge of the peripheral zone.

In still another aspect, the present invention also provides a method of minimizing the occurrence of pseudophakic pupillary block in a patient implanted with an IOL, including, providing an IOL including a peripheral zone disposed around an optical zone, the optical zone having an anterior optical zone face and a posterior optical zone face and at least one channel formed through the optical zone at an angle in the range of zero degrees to twenty degrees from a line parallel to a horizontal reference line drawn across the peripheral edge of the peripheral zone, the channel having an anterior opening and a posterior opening and implanting the IOL in the eye of the patient.

Other features and advantages of the invention will become more apparent from the following detailed description of preferred embodiments of the invention, when taken in conjunction with the accompanying exemplary drawings.

### BRIEF DESCRIPTION OF THE DRAWINGS

These and other features, aspects and advantages of the present invention are described with reference to drawings of a preferred embodiment, which are intended to illustrate, but not to limit, the present invention.

FIG. 1 is a top plan view of an intraocular lens including channel openings.

FIG. 2 is a cross sectional elevational view of the intraocular lens of FIG. 1 taken through line A.

FIG. 3 is an enlarged schematic cross sectional view of an optical zone of the intraocular lens of FIG. 1 illustrating a configuration of channels through the intraocular lens.

FIG. 4 is a plan view of another embodiment of an intraocular lens in accordance with the present invention.

FIG. 5 is a plan view of another embodiment of an intraocular lens in accordance with the present invention, including curvilinear channel openings.

FIG. 6 is a plan view of another embodiment of the lens of FIG. 5.

FIG. 7 is a sectional view of the intraocular lens of FIG. 6 taken through line B.

FIG. 8 is an enlarged view through a peripheral edge portion of the intraocular lens of FIG. 6.

FIG. 9 is a representation of the phenomenon of total internal reflection, light lock, or optical lock.

### DETAILED DESCRIPTION OF THE INVENTION

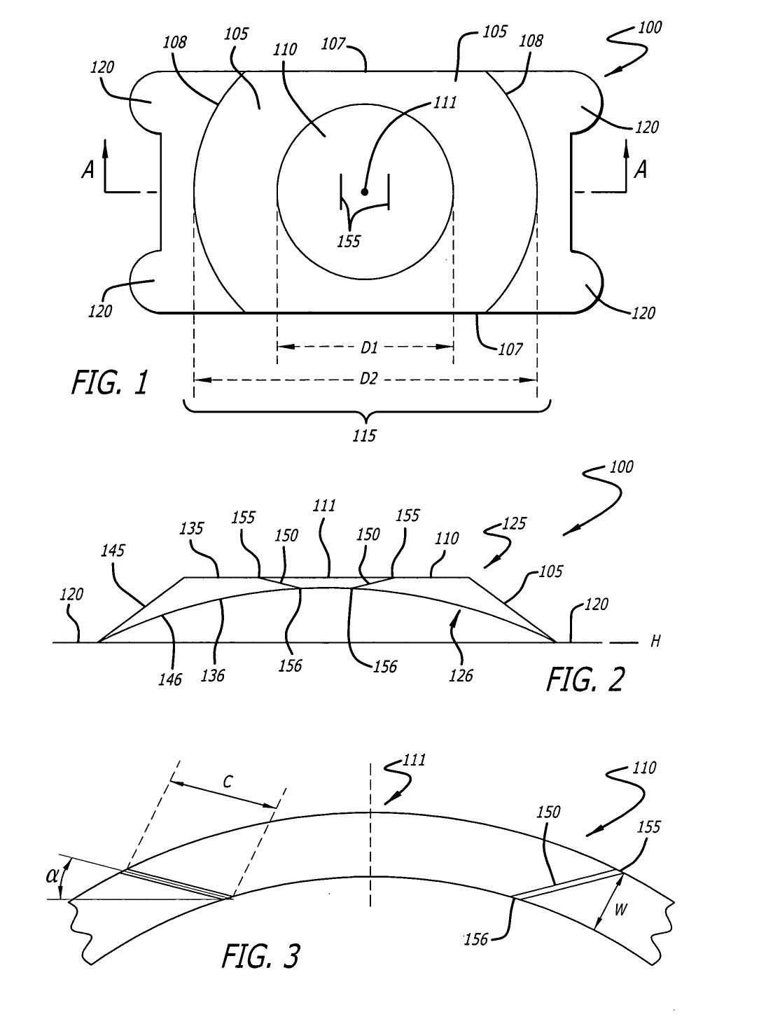

Referring to the drawings, which are provided for purposes of illustration and by way of example, one or more embodiments of the present invention of an intraocular lens (IOL) are illustrated in FIGS. 1-8.

Referring first to FIGS. 1-3, in general terms, there is provided in the present invention an intraocular lens (IOL) 100. The IOL includes an optic 115, having a peripheral zone 105 circumscribing an optical zone 110. In at least one embodiment, the IOL further includes one or more haptics or fixation members 120 that are coupled to the peripheral zone and extend outwardly from the peripheral zone to assist in retaining the optic in position in an eye of a patient.

In one embodiment the fixation member 120 or members are preferably located so that the optical zone 110 is free of such member or members. The peripheral zone 105 preferably forms, in effect, a frame which assists in strengthening the optic 115 against unwanted deformation after implantation in the eye. The peripheral zone may include buttresses for use in attaching the fixation members to the optic and for lending support to the optic. The fixation members may be one or more of several types known in the art and discussed in further detail below. In one embodiment, the peripheral edge of the peripheral zone may be beveled (FIG. 8), for example, at a 45 degree angle.

Referring more specifically to FIG. 2, the optic 115 has an optical axis and further includes an anterior surface 125 and a posterior surface 126. The optical zone 110 has an anterior optical zone face 135 and a posterior optical zone face 136. The curvature of one or both of these optical zone faces determines the corrective or diopter power of the optic. The peripheral zone 105 has an anterior peripheral face 145 and a posterior peripheral face 146.

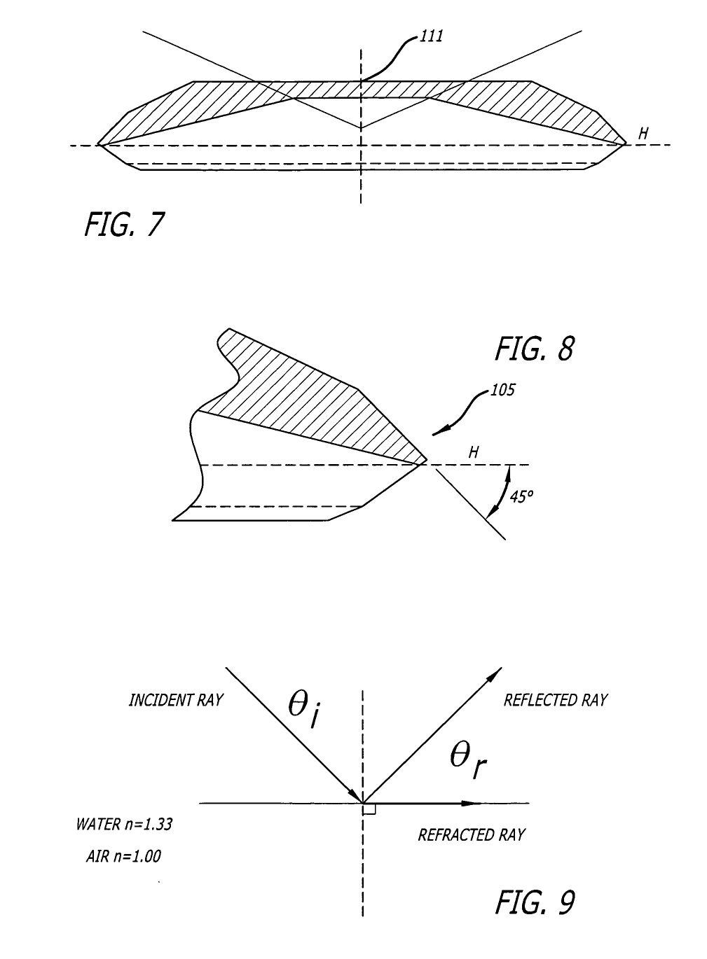

Referring more specifically now to FIG. 1, the optical zone 110 has a diameter D1 having a center 111. The peripheral zone 105 has a diameter D2, also having the center 111. The peripheral zone may have two long sides 107 and two short sides 108. In at least one embodiment, the peripheral zone may be configured as two generally parallel long sides 107 having a length L2 and two other curvilinear sides 108 having a diameter D2. As shown in FIGS. 7-8, in one embodiment the circumferential edge of the peripheral zone may be configured with a forty-five degree angled bevel.

Again referring to FIGS. 1-3, the intraocular lens 100 includes one or more channels 150 having an anterior opening 155 in the anterior optic surface 125 of the IOL and a posterior opening 156 in the posterior optic surface 126 of the IOL. In one embodiment, the channels are slit-like in configuration. The channel 150 permits a controlled flow of fluid, for example, aqueous humor, through the lens between the posterior optic surface 126 and the anterior optic surface 125. In one embodiment, the anterior opening and/or the posterior opening of the channel is slit-like in configuration. In one preferred embodiment, the channel and slit-like openings are disposed in the optical zone 110, thereby permitting fluid, for example, aqueous humor, to flow from the posterior optical zone face 136 through the anterior optical zone face 135.

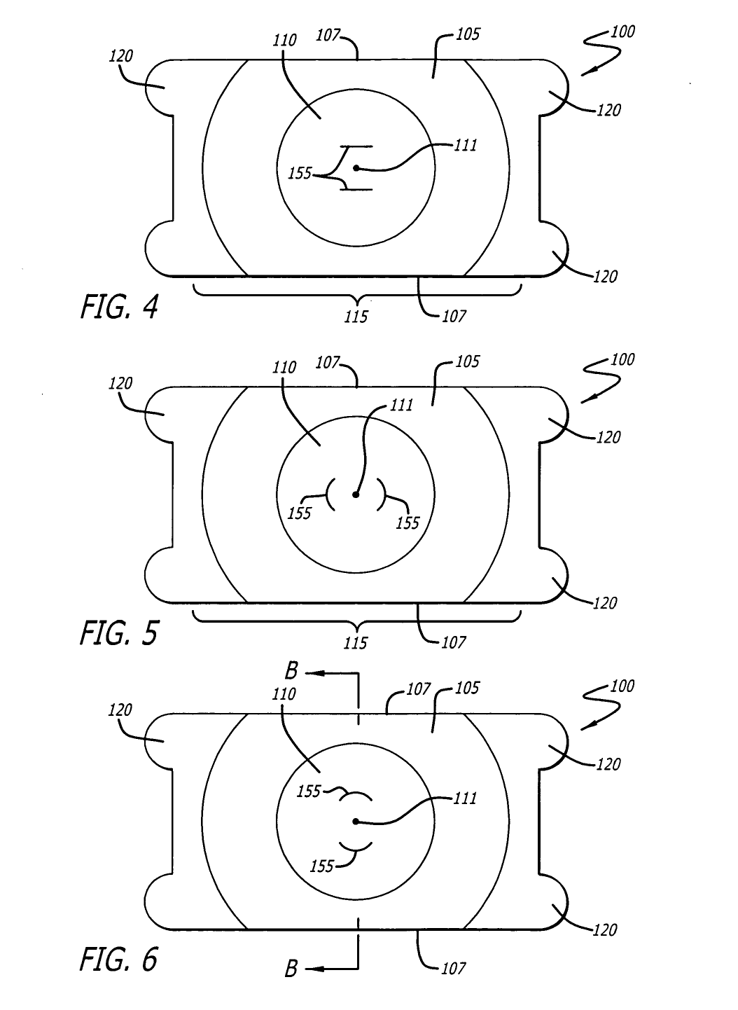

In another embodiment of the present invention, the channel 150 is angulated as it traverses across the optic 115, wherein the posterior opening 156 of the channel is disposed closer to a center 111 of the optical zone 110 than the anterior opening 155 of the channel. In one embodiment (FIG. 1), the slit-like openings of the channel may be configured substantially parallel to the generally parallel long sides 107 of the peripheral zone 105. In at least one other embodiment, the slit-like openings of the channel may be configured substantially perpendicular to the generally parallel long sides of the peripheral zone (FIG. 4). The channel may be configured with straight walls and/or openings (FIG. 1 and FIG. 4) or with curvilinear walls and/or openings (FIGS. 5-6). The curvilinear openings may be configured substantially perpendicular to the long sides of the peripheral zone (FIG. 5) or substantially parallel to the long sides of the peripheral zone (FIG. 6).

Referring more specifically now to FIG. 3, the channels 150 and openings 155, 156 provide an optically clear and distortion-free valve for the intraocular lens 100. In one preferred embodiment, the channels and openings are made completely through the optic zone 110 of the intraocular lens and are configured at a critical angle that produces an optical lock or light lock. A perpendicular cut in the lens would not reflect light and the patient would see a distorted image. The critical angle of the cuts produces a light lock or optical lock wherein light does not pass through the valves. In at least one embodiment, the critical angle is in the range of zero to twenty degrees. The critical angle α of the cut results in a light lock or optical lock that prevents light from passing through the valve. The angle of the channel may be defined by the formula:
α=arc sin(W/C)
where α is the angle of the channel in relation to a line parallel to a horizontal reference line H (FIG. 2), W is the width of the optical zone 110 at the level of the anterior opening, and C is the length of the channel 150 from anterior opening 155 to posterior opening 156. As shown in FIG. 2, horizontal reference line H is a line drawn across the circumferential edge of the peripheral zone 105 in a frontal view.

Referring briefly now to FIG. 9, total internal reflection (TIR), light lock, or optical lock is the phenomenon which involves the reflection of all the incident light off the boundary. TIR only takes place when both of the following two conditions are met: 1) a light ray is in the more dense medium and approaching the less dense medium and 2) the angle of incidence for the light ray is greater than the so-called critical angle.

One example of the phenomenon of TIR is light traveling through water towards the boundary with a less dense material such as air. When the angle of incidence in water reaches a certain critical value, the refracted ray lies along the boundary, having an angle of refraction of 90 degrees. This angle of incidence is known as the critical angle and it is the largest angle of incidence for which refraction can still occur. For any angle of incidence greater than the critical angle, light will undergo total internal reflection. For example, for the exemplary water-air boundary, at the critical angle the light will completely reflect off of the water-air boundary back into the water and will not penetrate into the air.

The critical angle is therefore defined as an angle of incidence which provides an angle of refraction of 90 degrees. The critical angle is an angle of incidence value. The actual value of the critical angle is dependent upon the combination of materials present on each side of the boundary. For example, for the water-air boundary, the critical angle is 48.6 degrees and for the crown glass-water boundary, the critical angle is 61.0 degrees.

The critical angle may be calculated for the boundary of two different materials. Medium “i” is the incident medium, and medium “r” is the refractive medium. The critical angle is the incident angle θᵢ which gives a refractive angle θᵣ value of 90 degrees. If this information is substituted into Snell's Law equation, a generic equation for predicting the critical angle can be derived. The derivation is shown below where “n” is the index of refraction.
nᵢ*sine(θᵢ)=nᵣ*sine(θᵣ)
nᵢ*sine(θ_{critical})=nᵣ*sine(90 degrees)
nᵢ*sine(θ_{critical})=nᵣ
sine(θ_{critical})=nᵣ/nᵢ
θ_{critical}=sine⁻¹(nᵣ/nᵢ)=invsine(nᵣ/nᵢ)

The critical angle can be calculated by taking the inverse-sine of the ratio of the indices of refraction. The ratio of nᵣ/nᵢ is a value less than 1.0. In fact, for the equation to even give a correct answer, the ratio of nᵣ/nᵢ must be less than 1.0. Since total internal reflection or light lock only occurs if the refractive medium is less dense than the incident medium, the value of nᵢ must be greater than the value of nᵣ. This equation for the critical angle can be used to predict the critical angle for any boundary, provided that the indices of refraction of the two materials on each side of the boundary are known. For the present invention, the critical angle of the valves is calculated from the index of refraction of the optic zone 110 materials and the index of refraction of the aqueous humor of the human eye.

Referring again to FIGS. 1-8, in at least one embodiment, there is a center symmetry in the positioning and configuration of channels 150 and openings 155. While other types of cuts or vents might permit aqueous humor flow through an intraocular lens 100, the present invention includes critical angle based angulations and locations of the cuts into the optical zone 110 that eliminate optical distortion and reflection from the surface of the channels and openings. In one embodiment, the invention includes channels and openings in the optical zone that are arranged symmetrically around the center 111 of the optical zone of the lens. Symmetrical arrangement of the cuts may result in better optical properties for the lens. However, in another embodiment, the channels and openings do not need to be arranged symmetrically around the center of the optic zone. The cuts may be made by mechanical means, for example, with a knife. The openings and channels may also be made with a laser or with water under high pressure directed at the IOL.

The channels and openings may be arranged symmetrically around a center 111 of the optic 115 of the lens. In at least one embodiment, a configuration of the channels 150 and openings 155, 156 will create an optical trap for light that may be coming over the openings and channels. The critical angle “α” of the channel can be calculated based on the anatomy of the human eye and a refractive index of the intraocular lens material. The critical angle “α” of the channel in one embodiment is preferably in the range of zero to twenty degrees to create the optical lock. The length of the channel may be calculated from the formula α=arc sin(W/C), where α is the angle of the channel in relation to a line parallel to a horizontal reference line H (FIG. 2), W is the width of the optical zone 110 at the level of the anterior opening, and C is the length of the channel 150 from anterior opening 155 to posterior opening 156. The width of the channel may be configured so that the flow through the channel will be in the range of four to five microliters per minute given the critical angle and length of the cut. The channel is further configured to maintain a patient's intraocular pressure in the non-pathologic normal range of 10 to 21 mm Hg.

In one preferred embodiment of the invention, the channels 150 and openings 155, 156 are configured to support a flow of fluid of five to six microliters per minute. This flow rate is important in preventing the build up of abnormal intraocular pressures and the development of glaucoma. Compression of the soft intraocular lens 100 of the invention during accommodation may cause the soft IOL to flex in response to changes of the ciliary muscles that may also promote pulsatile flow through the channel and between the two openings. This is advantageous for self cleaning of the openings.

The intraocular lens 100 of the present invention may be fashioned from the many materials well known in the art for making IOLs. These materials are preferably completely biologically and chemically inert. Many of these materials have also been used successfully as IOLs for cataract surgery, and have proven their safety within the eye. For example, the IOL of the present invention may be fashioned from, for example and not limited to, silicone, HEMA (2-hydroxyethylmethacrylate), Collamer® (STAAR Surgical Company), polydimethylsiloxane and PMMA (polymethylmethacrylate).

Furthermore, the optic 115 of the IOL 100 of the present invention may be made from materials known in the art for use in optics, for example, acrylic, biocryl, PMMA (polymethylmethacrylate), PMMA-heparin (polymethylmethacrylate-heparin), silicone, or collamer. The fixation member 120 may be made from materials known in the art for use in haptics, for example, acrylic, biocryl, collamer, PES (polyethersulfone), PMMA (polymethylmethacrylate), polyimide, polymide, polypropylene, PVDF (polyvinylidene fluoride), silicone, and the like. In at least one embodiment the IOL 100 may be made from silicone, covalently-bonded and UV absorbing (10% transmission at 395 nm). This material is completely biologically and chemically inert.

The optic 115 design may include shapes that are biconcave, biconvex, concavo-convex, convex-plano, convexo-concave, equiconvex, meniscus, plano-concave, plano-convex, planoconcave-convex, reverse-biconvex, or toric-spherical.

The haptics or fixation members 120 may be selected from various designs known in the art, for example, three point fixation, four point fixation, C-loop, Cap-C, circle, closed loop, CM loop, notched C, custom, foot plate, modified C-loop, modified J-loop, modified L-loop, modified S-loop, plate, short C, spiral loop, or Z-haptic.

The construction of the IOL incorporating aspects of the present invention may be one-piece or three piece. The IOL may be placed in the anterior chamber or in the posterior chamber of a patient's eye. In at least one embodiment, IOL may be foldable. In at least one other embodiment, the IOL may not be foldable. In one embodiment, the invention includes a deformable optic configured to enable the IOL to be passed through an incision, for example, a scleral tunnel incision into the eye.

In one embodiment, the length of the intraocular lens 100 is in the range of 8.5 to 14.5 millimeters. In another embodiment, the optic 115 size is in the range of 3.50 to 7.00 millimeters. In yet another embodiment, the power of the optic is in the range of −40.0 to +45.0 diopters. In still another embodiment, the power of the optic may vary in 0.5 diopter increments.

In one embodiment, the A-constant, used in calculating the power of the IOL needed to replace the removed cataractuous lens, is in the range of 114.0 to 120.0.

In yet another embodiment, the invention includes a method of making a distortion-free IOL configured with a valve. The method includes forming a lens from a biocompatible material, the lens including a peripheral zone disposed around an optical zone, the optical zone having an anterior optical zone face and a posterior optical zone face, and the peripheral zone having an anterior peripheral face and a posterior peripheral face. Examples of suitable biocompatible materials are included above. The invention further includes forming at least one channel through the optical zone at a critical angle that provides optical lock or light lock. In one embodiment, the angle is formed at an angle in the range of zero degrees to twenty degrees from a line parallel to a horizontal reference line drawn across the circumferential edge of the peripheral zone. In one embodiment, the channel is formed using a mechanical device. In one other embodiment, the channel is formed using a laser. In yet another embodiment, the channel is formed using a jet of water under pressure. Other methods of forming channels in an IOL may be used without departing from the intended scope of the invention. The method may further include disposing at least one fixation member on the IOL.

The invention also includes a method of minimizing the occurrence of pseudophakic pupillary block in a patient implanted with an IOL, including providing an IOL including a peripheral zone disposed around an optical zone, the optical zone having an anterior optical zone face and a posterior optical zone face and at least one channel formed through the optical zone at a critical angle, the channel having an anterior opening and a posterior opening and implanting the intraocular lens in the eye of the patient. In one embodiment, the lens that is provided includes channels configured at a critical angle in the range of zero degrees to twenty degrees from a line parallel to a horizontal reference line drawn across the peripheral edge of the peripheral zone.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

We claim:

1. An intraocular device for implantation in an eye of a patient, comprising:
an optical zone, having an anterior optical zone face and a posterior optical zone face;
a peripheral zone disposed around the optical zone, the peripheral zone having an anterior peripheral face and a posterior peripheral face;
an anterior opening in the anterior optical zone face;
a posterior opening in the posterior optical zone face; and
a slit shaped channel disposed between the anterior opening and the posterior opening.

2. The intraocular device of claim 1, wherein the lens has two channels, and the two channels are symmetrically disposed around a center of the optical zone.

3. The intraocular device of claim 1, wherein the channel is configured at an angle of less than ninety degrees from a line parallel to a horizontal reference line drawn across the circumferential edge of the peripheral zone.

4. The intraocular device of claim 1, further including at least one fixation member disposed on the peripheral zone.

5. The intraocular device of claim 1, wherein the posterior opening of the channel is configured closer to a center of the optical zone than the anterior opening of the channel.

6. The intraocular device of claim 1, wherein the channel is configured at an angle in the range of zero degrees to twenty degrees from a line parallel to a horizontal reference line drawn across the circumferential edge of the peripheral zone.

7. The intraocular device of claim 1, wherein the channels are configured to support a flow of fluid of five to six microliters per minute.

8. The intraocular device of claim 1, wherein the channels are configured to maintain a patient's intraocular pressure in a non-pathologic range.

9. The intraocular device of claim 1, wherein the channels are configured to maintain a patient's intraocular pressure in a range of 10 to 21 mm Hg.

10. The intraocular device of claim 1, wherein the intraocular device is made from a biocompatible material.

11. The intraocular device of claim 1, wherein the intraocular device is made from a flexible material.

12. The intraocular device of claim 1, wherein the channels and openings in the channels are configured to provide an optical lock.

13. A method of making a distortion-free intraocular lens configured with a valve, comprising:
forming a lens from a biocompatible material, the lens including a peripheral zone disposed around an optical zone, the optical zone having an anterior optical zone face and a posterior optical zone face, and the peripheral zone having an anterior peripheral face and a posterior peripheral face; and
forming at least one channel through the optical zone at a critical angle that provides optical lock or light lock.

14. The method of claim 13, wherein the channel is formed using a mechanical device.

15. The method of claim 13, wherein the channel is formed using a laser.

16. The method of claim 13, wherein the channel is formed using a jet of water under pressure.

17. The method of claim 13, further including disposing at least one fixation member on the intraocular lens.

18. The method of claim 13 wherein the critical angle is in the range of zero degrees to twenty degrees from a line parallel to a horizontal reference line drawn across the circumferential edge of the peripheral zone.

19. The method of claim 13, further including calculating the critical angle of the lens from the index of refraction of the lens material and the index of refraction of aqueous humor.

20. A method of minimizing the occurrence of pseudophakic pupillary block in a patient implanted with an intraocular lens, comprising:
providing an intraocular lens including a peripheral zone disposed around an optical zone, the optical zone having an anterior optical zone face and a posterior optical zone face and at least one channel formed through the optical zone at an angle in the range of zero degrees to twenty degrees from a line parallel to a horizontal reference line drawn across the peripheral edge of the peripheral zone, the channel having an anterior opening and a posterior opening; and
implanting the intraocular lens in the eye of the patient.
